(19) United States
(12) Patent Application Publication
(10) Pub. No.: US 2024/0105235 A1
(43) Pub. Date: Mar. 28, 2024

(54) METHOD AND SYSTEM FOR SUPPORTING AN EVALUATION OF A VIDEO-ASSISTED MEDICAL INTERVENTIONAL PROCEDURE

(71) Applicant: Erbe Elektromedizin GmbH, Tuebingen (DE)

(72) Inventor: Peter Selig, Nehren (DE)

(73) Assignee: Erbe Elektromedizin GmbH, Tuebingen (DE)

(21) Appl. No.: 18/373,123

(22) Filed: Sep. 26, 2023

(30) Foreign Application Priority Data
Sep. 27, 2022 (EP) 22198051.9

(51) Int. Cl. G11B 27/10; G06T 7/00; G11B 27/036; G16H 30/20; H04N 19/86

(52) U.S. Cl. CPC G11B 27/10; G06T 7/0012; G11B 27/036; G16H 30/20; H04N 19/86; G06T 2207/10016; G06T 2207/30004

(57) ABSTRACT

Methods and systems for supporting evaluation of video-assisted medical interventional procedure are disclosed and include receiving medical video image data (S1) representing video images of an examined or treated anatomy recorded by a video camera at a specific frame rate during a medical intervention; receiving further data (S2) comprising at least one of treatment or examination, device, diagnostic and measurement data associated with the medical intervention, the further data including measured dynamic data that varies during a frame period; identifying target areas in the video images for embedding the further data (S3); and modifying the video image data (S4) by embedding the further data into the video image data synchronously in time by replacing video image data in the identified target area or areas of a frame with the further data associated with the respective frame.

### CROSS-REFERENCE TO RELATED APPLICATION

This application claims priority to European Patent Application No. 22198051.9, filed Sep. 27, 2022, the entirety of which is incorporated by reference herein.

### TECHNICAL FIELD

The present invention relates to a system and method for supporting evaluation of a video-assisted medical interventional procedure. In particular, the invention relates to such methods and systems that are capable of simultaneously recording video image data from a medical intervention and further data associated with the intervention, including measurement data, in order to be able to subsequently evaluate the video image and further data.

Image data collected during a video-assisted medical intervention can provide a lot of valuable information, e.g., for the patient or the attending physician. It can be used for quality proofing and quality assurance of the treatment or examination performed, including for research, documentation and evaluation of the measures performed, facilitate the patient's understanding of the treatment or examination performed and can be included as a basis for post-operative treatment. In the case of such interventions, further information can also be of interest for the evaluation of a video-assisted medical interventional procedure, which may include, for example, information concerning the treatment or examination as such, its type, the patient or the attending physician, information concerning the medical devices used, diagnostic and/or measurement data obtained during the medical intervention. All this additional information can provide further insights of great value for the subsequent revue, traceability and evaluation of the medical treatments and/or examinations performed in conjunction with the recorded video data.

A problem in this context concerns the appropriate storage of all this information or data. In practice, there are currently no suitable procedures and systems that would enable the video image data to be stored in a suitable manner with a temporal relationship to the further data for an efficient subsequent evaluation of a video-assisted medical interventional procedure. In practice, established systems in hospitals are generally used for storing videos, whereas such systems for device, diagnostic or measurement data are lacking in the hospital environment. Device data can sometimes be stored in separate systems that are not a standard system in a hospital and are also not located within the direct access of the hospital, but are accommodated, for example, at the manufacturer of the respective medical device.

When data from two different systems needs to be merged, the issue of temporal allocation and synchronization is critical. Exact synchronization of, for example, electrical data from an RF surgery to individual video frames of the recorded video image data is practically impossible. In addition, separate systems make it difficult to comply with data protection and data security.

DE 10 2014 210 079 A1 describes a system for documentation of operations, in which medical image material is provided with a security feature, e.g., a digital watermark, which is integrated into the image data without being visible. In this way, further static information, such as type, serial number of the medical device, certificates, name of the patient and the attending physician, and the like, can also be integrated into the image data.

U.S. Pat. No. 6,104,948 A discloses a method and system for visually integrating multiple data acquisition technologies for real-time analysis, where data from different sources, such as date, time, electrophysiological data and video data, is combined in a computer and displayed together, side-by-side on a monitor. The combined image displayed on the monitor can then be stored.

WO 2012/162427 A2 discloses a general system for embedding metadata in video content and television broadcasts. The metadata is embedded in the transmitted video or television images in the form of QR codes in order to enable a viewer to quickly and preferably automatically decode a string of characters contained in the metadata, such as a command set or link.

EP 2 063 374 A1 discloses a method and a device for documentation of medical data, in which, in addition to video data, for example, endoscopic image data, further information, including data about the type, location, function and/or parameters of the instruments or devices used, data on the pressure and flow of an insufflation gas and related measured values, e.g. pressure, leakage current, temperature, or also anesthesia data and vital parameters can be stored in order to enable the most complete possible documentation of the processes during an operation. To limit the amount of data recorded, storage is only activated when the presence of a patient to be operated on is detected in the operating room.

WO 03/001359 A2 discloses a system for the simultaneous recording of measurement data and video/audio data, in particular for the documentation of psychological therapy sessions, whereby the heart rate, for example, is recorded in addition to video data. Surgical and diagnostic data can also be documented. For simultaneous recording of the measurement data and video and/or audio data, both the measurement data and the video and/or audio data are broken down into data packets of the same packet length and stored in a common data file in an orderly manner. The video and/or audio data is first compressed, divided into packets and combined with the packets of the measurement data.

WO 2006/131277 A1 discloses a system for diagnosing, annotating and/or documenting moving images in the medical field, such as ultrasound images or endoscopic video recordings. Annotations, such as markings, comments, images, text, audio, etc., are included in the images or videos and played along at the appropriate position when the video is replayed.

WO 2014/181230 A2 discloses a method for embedding encoded information, e.g., image parameters and diagnostic information, into medical images. The information is burned into the image data or stored on a separate layer, such as a DICOM overlay layer, over the image data. In embodiments, the information is positioned in the form of a barcode or QR code in an area of an image outside of the visualized anatomy or along the outer edges of the image.

EP 3 836 155 A1 discloses the integration of information, e.g., patient data, metadata or features extracted from deep learning or neural networks, into medical diagnostic images. The information can be encoded in barcodes or QR codes, overlaid on the images or transparently merged with the images, and is displayed together with or alternating with the images.

The aforementioned prior art systems and methods suffer from various shortcomings. Either they do not allow synchronous assignment of further data, such as examination, device and measurement data, to recorded video image data, or they require a relatively high level of effort to ensure time-synchronous storage and synchronization of the video and further data.

In addition, there are limits to the systems and methods in terms of evaluation possibilities. For example, in radiofrequency (RF) surgery, the relationships between the treatment effects and the set variables, parameters and selected application modes, the effectiveness of the desired treatment effects and the like can only be insufficiently evaluated on the basis of the recorded video image data and the stored static treatment and device data. In particular, many processes involving, for example, the generation of sparks on the RF surgical instrument, the introduction of a high-frequency alternating current through the human body and its interaction with the treated tissue, the heating of the tissue caused thereby and the resulting cutting or coagulation effects are very fast and time-critical processes that are not easy to assess in this manner. There remains a need for systems and methods that overcome the shortcomings of conventional systems and methods.

Based on the above, it is an object of the present invention to provide a method and system for supporting evaluation of a video-assisted medical interventional procedure, that allow a time assignment and synchronization of video image data and further data with relatively little effort for storing the data. In particular, the system and the method should enable reliable retrospective evaluation of the medical interventional procedure, including time-critical processes, especially of a video-assisted RF surgical intervention.

This object is achieved by the method and the system for supporting evaluation of a video-assisted medical interventional procedure, which have the features of the independent claims 1 and 17. Particularly preferred embodiments of the invention are the subject-matter of the dependent claims.

According to one aspect of the present invention, a method for supporting evaluation of a video-assisted medical interventional procedure is provided, the method comprising the steps: receiving medical video image data representing video images of an examined or treated anatomy recorded by a video camera at a specific frame rate during a medical intervention; receiving further data comprising at least one of treatment or examination, device, diagnostic and measurement data associated with the medical intervention, the further data including measured dynamic data that varies during a frame period; identifying target areas in the video images for embedding the further data; and modifying the video image data by embedding the further data into the video image data synchronously in time by replacing video image data in the identified target areas of a frame with the further data associated with the respective frame.

The method according to the invention enables time-synchronous recording of video image data and associated additional information that originate from a video-assisted medical intervention, a medical treatment, examination or operation. The method is preferably performed online, in parallel with the performance of the medical intervention, and in real time or at least near real time. The medical intervention can in particular be an RF electrosurgical interventional procedure, such as coagulation, thermofusion, RF cutting (electrotomy), and/or an examination, such as an optical emission spectrometry or impedance spectroscopy examination. The information may also relate to a therapy, such as RF therapy, cryotherapy, or waterjet therapy, and associated devices, as well as other system components, such as smoke evacuation equipment, and/or other apparatuses, instruments, and equipment in an operating room, such as the patient monitor, an operating table, and the like, all of which can be networked with one another via a data interface so that their data can also be acquired and stored in the video image data.

By directly embedding the treatment or examination, device, diagnostic and/or measurement data straight into the video images that document the progress of a video-assisted medical intervention, a high level of time synchronicity between the video and other data can be ensured with reduced storage requirements, and an extremely simple and effective evaluation of all data is made possible. In particular, the measured dynamic data, such as the measured values of electrical RF variables, which can change at very high frequency and in particular within each frame, can be stored along in the correct associated frame, which provides the basis for an effective evaluation of time-critical processes of an RF surgical procedure, for example. For example, a good assessment of spark formation, including the ignition behavior, the spark intensity or a spark breakaway, in an RF cutting or RF coagulation procedure can be made possible in this way.

The resulting modified video image data can be stored in a non-volatile storage device for further use, in particular for evaluation. In particular, the storage device may be arranged in the vicinity of the place where the medical interventional procedure was performed, e.g., in the respective hospital. A memory in a computer or a computer system or a server or even a cloud storage can serve as a storage device.

In a particularly preferred embodiment, the modified video image data is transmitted alternatively or additionally to an image archiving and communication system, so-called Picture Archiving and Communication System (PACS), a clinical information system (CIS), also known as a Hospital Information System (HIS), or an external server for archiving image/video data, which can be accessed for evaluation purposes by the hospital in which the medical intervention was performed and/or by the manufacturer of the medical device used.

The video camera intended for the video recording of the medical procedure delivers the individual images with a specific frame rate, also called refresh rate, which corresponds to a specific number of frames (individual images) per second. The frame rate can be between 24 and 60 frames per second (fps). It is preferably 30, 50 or 60 fps. Higher frame rates, such as 50 or 60 fps, are more preferred because then even very time-critical processes, such as those of interest in RF surgery, can be optically resolved with high resolution. However, the memory requirement increases with a higher frame rate.

The treatment or examination data may include, for example, the place and time of the intervention, the institution, persons involved, such as the name of the patient, the name of the attending physician, the type of medical intervention and/or other data relating to the medical intervention.

The device data may include, for example, a type designation and serial number of the medical device or devices used, device parameters, default settings, selected application mode, preset limit values for variables, error and status messages from the devices and/or other data relating to the medical devices used.

The diagnostic data may include, for example, determined spectra and/or classification results from an optical emission spectrometry, spectra, measured values and/or classification results from an impedance spectroscopy or from other diagnostic methods, and other diagnostic data.

The measurement data, in particular the dynamic measurement data, may include electrical RF variables measured or determined during an RF interventional procedure, such as voltage, current, power, power factor and/or spark intensity, e.g., in RF cutting and coagulation procedures, parameters or variables of a neutral electrode when performing monopolar RF techniques, such as transition impedance, current symmetry, and/or current density, and/or other dynamic measurement variables obtained during the medical intervention.

Instead of including this data in the video image in the raw format, the data can be encrypted and/or provided with a digital certificate to ensure authenticity and/or compressed to save storage space before incorporation into the video image.

In preferred embodiments of the invention, the measured dynamic data may include measured quantities that are acquired or determined at an update rate that is several times, at least two times or more, greater than the frame rate of the video image data. For example, the update rate can be at least 150 Hz, preferably 250 Hz or even more. Thus, several measured values of a dynamic measured variable can be acquired and stored per frame.

In preferred embodiments, measured data can thus advantageously be stored in individual frames, which characterizes a time course of two or more measured values of a measured variable. With a frame rate of, for example, 50 fps and an update rate of 250 Hz, for example, five measured values can be stored synchronously over time per frame of the video image data. The measurement data belonging to an individual frame is stored time-synchronously with the video image data, so that there is a fixed temporal relationship between the image information in the individual frame and the associated time course of the measurement variable.

The identification of target areas in the video images for embedding the further data can be easily done based on suitable areas in the video images that have been defined in advance or are specified or can be specified by a user. For example, the data pixels of the further data can be stored contiguously and can be put, for example, as a small strip or block at a defined position in the image. Preferably, they are stored at several defined positions, distributed over the image.

It is particularly preferred if the storage takes place in less relevant image areas, i.e., in image areas which have no or only little useful information content of the image data. These can be, for example, areas that are reserved for labeling, displayed patient data, time of day and the like, or even the outer image edges of the video images. This may preferably also be a non-visible area, if present, which is cut off for display.

Instead of using individual pixels of the video image data entirely for data storage, in an alternative storage procedure, an overlay of the image data with the original video pixel color can be applied. In other words, the further data can be incorporated into individual bits of a pixel color of a plurality of pixels of the video image data. This reduces the color depth because all of the bits are no longer available for the video image data. For example, instead of 8 bits per color channel (R, G, B) with 24 bits of useful information per pixel, the color depth can be reduced to 7 bits per color channel, while the remaining 3 bits per pixel are used to store or encode the further data. No part of the image is completely blended anymore, but only slightly adjusted in color. In order to ensure the required high quality of the intra-operative video images, it is also preferable here to store the data in less relevant image areas with reduced useful information content, for example in peripheral areas.

Since video image data usually has to be compressed in a lossy manner for archiving, in order to reduce the amount of data appropriately, it is preferable to select a representation of the data in the video images that is not so strongly influenced by common compression methods, such as H.264, H.265, AV1 or VP9, that the information contained can no longer be reconstructed. According to an advantageous further embodiment of the method according to the invention, the modified video image data is processed by a compression invariance enhancement procedure before a compression procedure is performed, in order to increase reliability of a reconstruction of the video image data after decompression while eliminating or significantly reducing coding errors or compression artifacts.

For example, the measured values or other additional data can be stored in a plurality of contiguous pixels, in particular by forming pixel blocks, e.g., 8×8, 16×16 or even up to 64×64 pixel blocks, in each individual image of the video stream. Relatively coarser structures of this type, which occupy more area in the image, are less prone to artifacts when they are compressed, so that the block formation increases robustness against compression artifacts.

In addition or as an alternative, common error correction methods can also be applied to the further data. This further reduces the effective amount of data, but it also increases the reliability of the reconstruction while avoiding compression artifacts.

The modification of the video image data and the compression of the modified video image data are preferably performed in a common signal processing device. The signal processing device can then perform the compression invariance enhancement procedure depending on the selected compression method and, if necessary, also check whether the data can be stored correctly, or dynamically adapt the structure size of the data to the respective compression.

In a further embodiment of the method, steps for evaluating the stored modified video image data can also be performed. These steps can include retrieving the stored modified video image data, extracting the further data from the modified video image data, and analyzing the extracted static data and the measured dynamic data, in particular electrical measurement data, in connection with the video image data to evaluate the medical intervention performed. In particular, the evaluation can include one or more of the following evaluation goals: assessment of the input variables or parameters set for the medical intervention, evaluation of the selected application modes, assessment of the effectiveness of the desired treatment effects, finding of optimization possibilities for the set variables, application modes or the medical device or devices, detection of inadequacies, including electrical limitations for the application, e.g. insufficient RF current or insufficient RF voltage for a cutting operation, detection of insufficient speed of treatment effects, e.g., in cutting, coagulation or thermofusion, detection of occurrence of lateral damage, such as carbonization, onset of bleeding, e.g., based on electrical quantities measured over time, and determination of causes of the lateral damage or other complications. These evaluation goals can be accomplished in particular by evaluating the dynamic electrical measurement data in conjunction with associated image information, such as a visually recognizable spark formation and intensity, an electrode or an instrument recognizable in the image, a tissue recognizable in the image and/or the transitions between different tissue types, visible bleeding or flushing with liquids, visible smoke development and/or vapor formation and heat generation during thermofusion.

The evaluation of the aforementioned relationships can be performed manually by an operator or automatically by using a suitable algorithm based on pattern recognition, deep learning, neural networks or even artificial intelligence. The knowledge gained can be useful for the treating surgeon for quality assurance, for future users and for the further development of medical RF devices.

According to a further aspect of the invention, a system for supporting evaluation of a video-assisted medical interventional procedure is provided, the system comprising: at least one medical device for performing a medical intervention, in particular an RF surgical device; a video camera for recording video image data representing video images of treated or examined anatomy during a medical intervention; a signal processing device for processing the video image data recorded by the video camera; and a data connection between the at least one medical device and the signal processing device for transmitting further data, including dynamic data, from the at least one medical device to the signal processing device. The signal processing device is configured to perform the method as described above.

The merging of examination, device, diagnostic and/or measurement data with the video image data thus takes place in the signal processing device, which is preferably implemented on a dedicated computing device, e.g., a computer or the like, separate from the medical treatment or examination device. The signal processing device could also be integrated into a medical device. In any case, the signal processing device preferably comprises interfaces to connect it to the medical device or devices, including the other system components and instruments and other equipment in the operating room, such as a patient monitor or an operating table.

The signal processing device is preferably configured to perform the method according to the invention online, while the medical intervention is being performed, in real time or near real time. However, the video image data and further data could in principle also be processed offline and/or subsequently in the manner according to the invention by the signal processing device, if they are temporarily stored in a suitable manner, e.g., using time stamps or the like, in such a way that a subsequent temporal assignment for synchronous combination of the video and further data is possible.

In a further embodiment, the system according to the invention can also be configured for downstream evaluation of the data. For this purpose, additional software or firmware with extended algorithms can be implemented, for example, in the signal processing device, which make it possible to extract the further data, in particular the static treatment or examination, device data and dynamic measured values, from the individual frames and make them available for analysis, or to automatically evaluate the data using an algorithm.

In all other respects, the explanations given above in connection with the method according to the invention regarding possible embodiments and their advantages equally apply to the system according to the invention, so that it is expressly referred thereto.

Further advantageous details of embodiments of the invention are apparent from the dependent claims, the drawing and the corresponding description. The drawing shows in no way limiting exemplary embodiments of the subject of the invention. In the drawing:

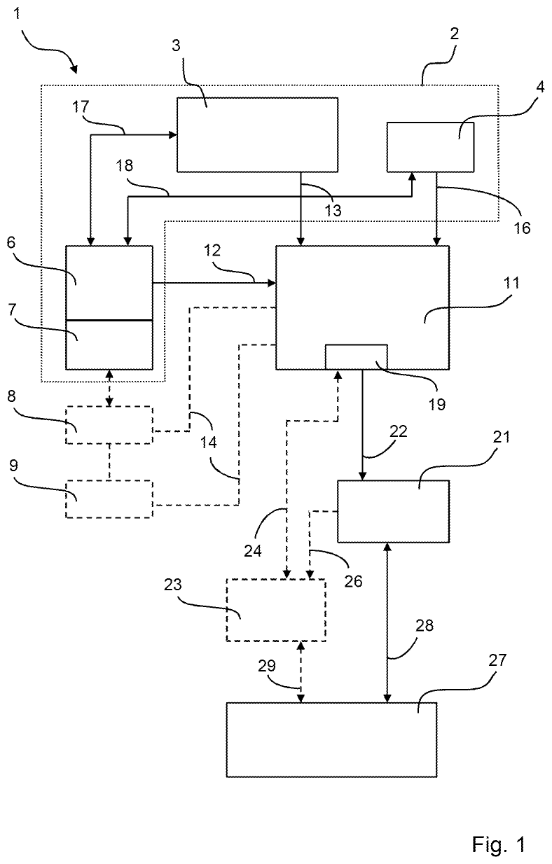

FIG. 1 shows an embodiment of a system for supporting evaluation of a video-assisted medical interventional procedure according to an embodiment of the invention;

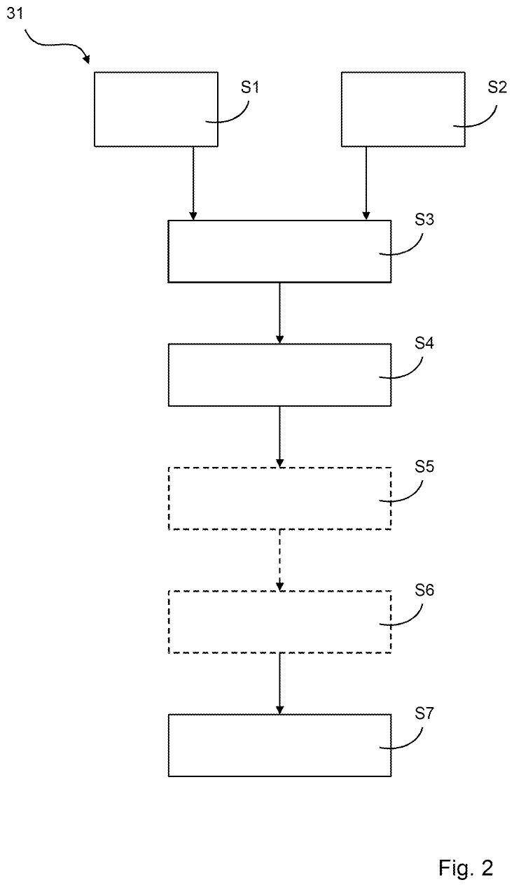

FIG. 2 is a flow chart of an exemplary method for supporting evaluation of a video-assisted medical interventional procedure according to an embodiment of the invention;

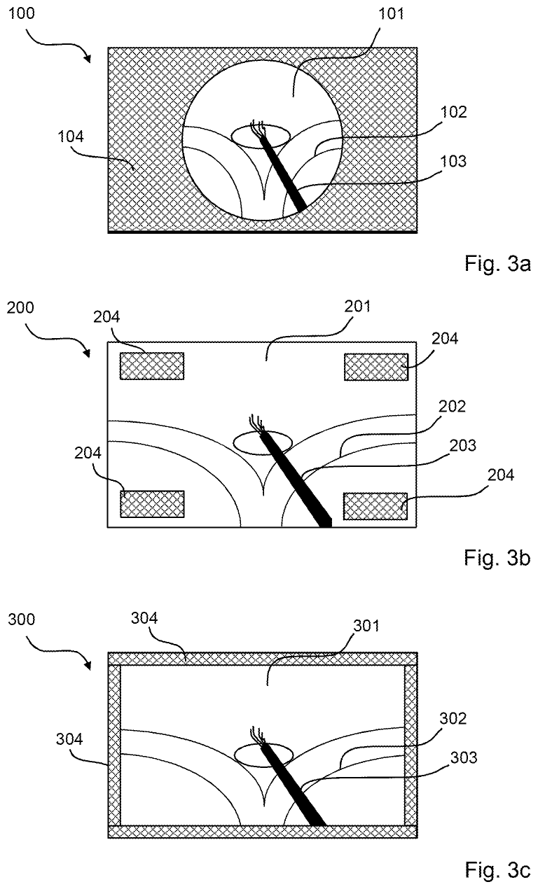

FIGS. 3a-3c are representations of individual video images from a medical interventional procedure to illustrate target areas for embedding further data in video image data, in a highly simplified representation;

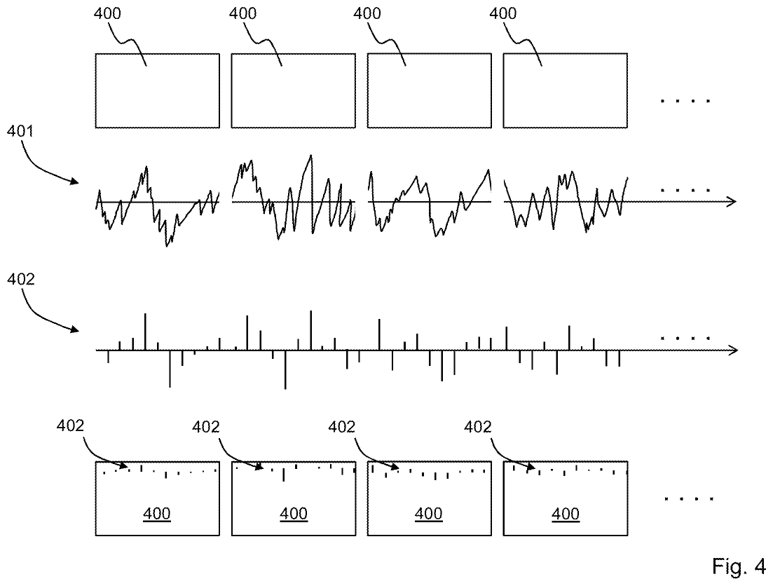

FIG. 4 is a schematic diagram for explaining the functioning or operation of the system and method according to the invention; and

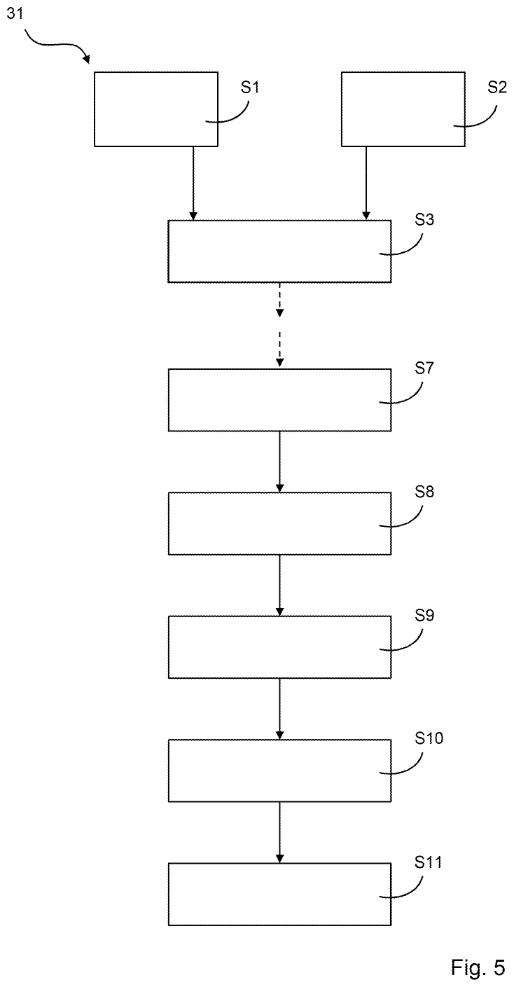

FIG. 5 shows a method for supporting evaluation of a video-assisted medical interventional procedure according to a further embodiment of the invention.

In FIG. 1, a system 1 for supporting evaluation of a video-assisted medical interventional procedure according to an embodiment of the invention is shown in a greatly simplified block diagram representation. The system 1 includes a medical treatment and/or examination device 2 which is arranged for examination or treatment, e.g. therapy or surgery, of patients. In preferred embodiments, the device 2 is an RF surgery device used for RF surgery.

The treatment and/or examination device 2 comprises a medical device 3, in particular an RF surgical device, which is used, for example, for RF cutting, coagulation, devitalization or thermofusion in open surgery or for laparoscopic diagnostic or therapeutic procedures, or an endoscopic RF device, for example, for argon plasma coagulation. Other medical devices, in particular therapeutic devices, such as RF, cryogenic or waterjet devices, or diagnostic devices, such as for optical emission spectrometry or impedance spectroscopy, to which the invention can be applied, are also contemplated.

The treatment and/or examination device 2 further comprises a video camera 4, which is configured to capture video images of an examined or treated anatomy during a medical intervention at a predetermined frame rate, such as 25 frames per second (fps), 30 fps, 50 fps or 60 fps. The higher the frame rate (refresh rate), the smoother videos can be played and details can be sharply reproduced even with high dynamics in the video frames or in slow motion.

Although the video camera 4 is shown separately from the medical device 3 in the system 1 shown in FIG. 1, it can also be integrated into the medical device 3, for example in endoscopic systems.

The treatment and/or examination device 2 further comprises a control device 6 which is connected to the medical device 3 to control or regulate the operation of the medical device 3. In particular, the control device 6 can include an RF generator to be able to generate, adjust and deliver to the medical device 3 the electrical quantities required for the operation of the medical device 3, such as RF voltages and RF currents. During operation, the control device 6 can control or regulate the magnitude of the electrical quantities, dynamically adjust the modulation frequency of the electrical quantities, and perform other control or regulation operations in order to cause the medical device 3 to perform the respectively desired examination or treatment operation.

The control device 6 may be connected to an input device, not shown in detail herein, which allows the user, for example an attending physician, to make the desired settings or select parameters. Preferably, the control device can allow a user to select from a plurality of predefined modes that are predetermined for specific applications and tailored to specific working instruments, such as different cutting, coagulation or vessel-sealing modes.

The control device 6 can also control the supply of gases, such as argon, or liquids, for example for rinsing tissue, to the medical device 3.

The control device 6 can also control the operation of the video camera 4, as indicated by the illustrated communication link between the video camera 4 and the control device 6 in FIG. 1

The control device 6 can also be configured to monitor a medical interventional procedure and, for this purpose, receive measurement data from the medical device 3 that corresponds to values measured or determined during operation. For this purpose, sensors may be provided on the medical device 3 which detect the respective variables, in particular electrical variables, during operation of the medical device 3. The measured variables may include RF electrical measured quantities, such as voltage, current, power, power factor, or spark intensity, which are measured in situ at the surgical site during operation or determined from other measured values. In the case of monopolar RF treatment or examination techniques, the measured variables may also include parameters or variables of a neutral electrode, such as transition impedance, current symmetry or current density, which can also be acquired by sensors or determined during the medical intervention.

The acquired data can be temporarily stored in a non-volatile memory 7, for example, a flash memory of the control device 6.

Although the control device 6 with the memory 7 are shown herein as separate units from the medical device 3, they can also be at least partially integrated into the medical device 3. Other configurations for the treatment and/or examination device 2 are also possible, which can deviate from the configuration shown in FIG. 1.

As may also be seen from FIG. 1, the system 1 may comprise further medical devices 8, 9. For example, a medical device 8 can be arranged to perform a different RF treatment than the medical device 3. For example, the medical device 3 can be arranged for coagulation or vessel sealing, while the medical device 8 is prepared and arranged for performing cutting operations. The control device 6 can be configured to alternately control, regulate and/or monitor both medical devices 3 and 8 during a medical intervention.

Yet another medical device 9 may represent another system component, for example for smoke evacuation, another instrument, another equipment in the operating room, such as a patient monitor, an operating table and the like, which may all be networked to the device 2. The data provided by the optional further medical devices 8, 9 can be linked and stored together with data from the medical device 3 or the control device 6 and video image data from the video camera 4 in order to be available for subsequent evaluation of a medical intervention.

In order to accomplish this, the system 1 further comprises a signal processing device 11, which is arranged to process the video image data recorded by the video camera 4 and further data supplied by the medical device 3 or the control device 6, as well as any data provided by the further medical devices 8 and 9, to merge them synchronously in time and to store them in a synchronized manner. For this purpose, the signal processing device 11 is communicatively connected to the other system components via at least one data connection.

In the exemplary embodiment shown in FIG. 1, a first data connection 12 is provided between the control device 6 and the signal processing device 11 in order to transmit data, in particular treatment or examination data, device data, diagnostic and/or measurement data, which can be acquired or generated during a medical interventional procedure and can be temporarily stored in the internal memory 7 of the control device 6, from the control device 6 to the signal processing device 11. Optionally, a second data connection 13 may additionally be established between the medical device 3 and the signal processing device 11 in order to be able to transmit data directly from the medical device 3 to the signal processing device 11 bypassing the control device 6. Such a configuration could be advantageous for sensor data that is particularly time-critical and is acquired at a high sampling rate. Further data connections 14 may be set up between the further medical devices 8, 9 and the signal processing device 11 for the same purposes.

Furthermore, in the embodiment according to FIG. 1, a video data connection 16 is provided between the video camera 4 and the signal processing device 11 for transmitting video data from the video camera 4 to the signal processing device 11

The data connections 12, 13, 14, 16 in the system 1 could be based on a bus system. Instead of the separate connections 12, 13 and 16, a single data connection 12 could also be provided between the control device 6 and the signal processing device 11, and all data from the medical device 3 and the video camera 4 could then be first transferred to the control device 6 via communication lines 17, 18 and forwarded from the control device 6 to the signal processing device 11 via the data connection 12.

The signal processing device 11 further comprises a data interface 19 arranged for data communication with external devices and equipment, such as an external storage device 21. The storage device 21 may be a server for archiving image, video, text and other data, a distributed storage system, a cloud-based storage system, or any other non-volatile storage capable of permanently storing the large amount of video image data generated during a medical interventional procedure. The storage device 21 is connected to the output-side data interface 19 of the signal processing device 11 via a data line 22 and receives thereover modified video image data from the signal processing device 11, i.e., video image data with further data embedded therein, for storage.

As can also be seen from FIG. 1, in a further embodiment of the system 1 according to the invention, the signal processing device 11 can, in addition to the storage device 21 or alternatively thereto, be communicatively connected to a hospital information system (HIS) comprising information processing systems for recording, processing and forwarding medical and administrative data included in a hospital. Thus, the modified video image data generated by the signal processing device 11 can be transmitted to the HIS 23 for further evaluation via the output-side data interface 19 and a communication link 24 and can be stored and made available in the HIS 23. The communication link 24 can be a wired or a wireless connection, for example a LAN or WLAN connection.

As an alternative, the HIS 23 may also be connected to the storage device 21 via a communication link 26 in order to be able to access it and retrieve relevant data therefrom.

Instead of a hospital information system, block 23 in FIG. 1 can also indicate a so-called Picture Archiving and Communication System (PACS), which is an image archiving and communication system based on digital computers and networks that is widely used in medicine. With a PACS, digital image data of different modalities in radiology, nuclear medicine and also images from other imaging procedures, e.g., endoscopy, cardiology, etc. can be acquired, processed and archived and sent to other viewing and post-processing computers as required.

In yet another embodiment, the system 1 may further comprise an analysis device 27 that may be connected to the storage device 21 via a data transmission connection 28 or to the HIS or PACS 23 via a data transmission connection 29 in order to be able to access the system 23 or the storage device 21, read data therefrom and analyze the data. In particular, the analysis device 27 may include software or firmware configured to extract the further data, such as treatment or examination data, device data, diagnostic data and measured values, from the stored modified video image data, provide them for analysis by a user or evaluate them automatically on the basis of an algorithm. Further details on the functionalities of the analysis device 27 are given below in connection with the method according to the invention shown in FIG. 5.

The system 1 described so far is for performing and supporting evaluation of a video-assisted medical interventional procedure. The medical intervention can in particular include an RF surgical procedure, such as coagulation, thermofusion, RF cutting (electrotomy), or another RF treatment. It can also include an examination, for example, by means of optical emission spectrometry or impedance spectroscopy, in which further data, in particular diagnostic data, is generated in addition to video image data. Still further, a therapy treatment, in particular an RF therapy, could also be performed using the system 1. The system 1 operates as follows:

During a video-assisted medical interventional procedure, the video camera 4 acquires video image data comprising video images of an anatomy being examined or treated at a specific frame rate, such as 30, 50 or 60 frames per second (fps) and transmits the video image data to the signal processing device 11 for further processing. In addition, the control device 6 and/or the medical device 3 transmits further data to the signal processing device 11. This further data can include treatment or examination data, device data, diagnostic data and/or measurement data that is related to the medical intervention. In particular, the further data also includes measured dynamic data that changes during a frame period.

The signal processing device 11 receives the video image data from the video camera 4 and the further data, including the dynamic measurement data, from the control device 6 and/or the medical device 3, identifies target areas in the video image data for embedding the further data, and modifies the video image data by embedding the further data into the video image data synchronously in time. This means that the signal processing device 11 replaces pixel data in the identified target areas of the frames with the further data associated with the respective frame.

Further details on the operation of the signal processing device 11 in connection with the time-synchronous recording of video image data and the further data are described in more detail below in connection with the method according to the invention as shown in FIG. 2.

The signal processing device 11 may transmit the generated modified video image data via the data interface 29 to the storage device 21 and/or the HIS or PACS 23 for short to long-term archiving. The analysis device 27 can access the archived modified video image data in order to subject them to an analysis to evaluate the medical intervention performed and the associated performance of the treatment and/or examination device 2.

Referring now to FIG. 2, a flow chart of a method 31 for supporting evaluation of a video-assisted medical interventional procedure in accordance with an embodiment of the present invention is shown therein. The method may be performed by the signal processing device 11 of the system 1 shown in FIG. 1, but is not limited to any specific signal processing device. Any computing unit having a processor and a main memory may be configured to perform the method 31 according to FIG. 2.

The method 31 of FIG. 2 assumes that a video-assisted medical interventional procedure is performed (not illustrated). The method 31 may then be performed online, while the video-assisted medical interventional procedure is being performed, in real time or near real time. It could also be performed subsequently, after the video-assisted medical interventional procedure has been performed, if all data is temporarily stored, although this is less preferred.

The method 31 starts with step S1 of receiving video image data. For example, the signal processing device 11 may receive the video image data from the video image camera 4 of the system 1 according to FIG. 1. As mentioned, the video image data is generated at a certain frame rate, preferably 30 fps, 50 fps, or 60 fps, in order to provide for good visual resolution of the treatment or examination procedures on the anatomy in the resulting video stream.

The method further comprises the step S2 of receiving further data. The further data may comprise one or more of treatment or examination, device, diagnostic and/or measurement data related to the medical intervention.

For example, the further data may comprise treatment or examination data, which may include the place and time of the procedure, the treating institution, the persons involved, such as the name of the patient, the name of the attending physician, the type of medical intervention and/or other data related to the medical intervention.

The further data may in particular comprise device data, which may include the type designation and/or serial number of the medical device or devices used, predetermined device parameters, default settings, selected application mode that automatically conditions certain device parameter settings and control of the electrical variables, specified limit values for variables, e.g., the electrical quantities, error and status messages generated by the medical devices during the medical interventional procedure, and other data pertaining to the devices or instruments used.

The optionally acquired diagnostic data may comprise spectra determined during an examination based on optical emission spectrometry and/or classification results therefrom. Alternatively, spectra, measured values and/or classification results from impedance spectroscopy or from other diagnostic procedures can also be received and processed.

The measurement data, in particular the dynamic measurement data, may comprise, in particular, electrical RF variables, such as voltage, current, power, power factor and/or spark intensity, which are acquired or determined, for example, in RF cutting and RF coagulation procedures. Parameters or quantities of a neutral electrode when using monopolar RF techniques, such as the transition impedance, current symmetry and/or current density, etc., can also be acquired or determined and processed.

The method 31 further comprises the step S3 of identifying target areas in the video images for embedding the further data.

Modern video systems work with a resolution of FullHD (1920×1080 pixels) or 4k (4096×2160 pixels) and a frame rate of 30 fps, for example. If 0.1% of the pixels of a video image are used to store device, diagnostic and measurement data, approximately 2k pixels per frame or approximately 62k pixels/s are available for FullHD and approximately 8.8k pixels per frame or approximately 265k pixels/s are available for 4k to embed the further data in the video image data. This results in a possible data volume of approximately 182k bytes/s for FullHD or 778k bytes/s for 4k. It has been determined that this amount of data is already sufficient for FullHD to store the further data in the video image data.

To store the raw data of optical emission spectroscopy (OES) or impedance spectra, a larger image area of approximately 1% would be required for FullHD, which is in principle practically feasible. If only the results of the classification are stored instead of raw data, a significantly smaller amount of data is sufficient.

In principle, the data pixels can be stored contiguously, for example, as a small contiguous strip or block, or they can be distributed at defined positions over each individual image. In step S3 the appropriate location or locations for embedding the data are identified. The identification may be based on suitable areas in the video images that have been defined in advance or are predefined or can be predefined by a user. Aa an alternative, a context recognition device could be configured to identify suitable areas on the video image to be displayed that are suitable for embedding the further data in the video image data.

FIGS. 3a-3c show exemplary video images 100, 200, 300, which reproduce individual images of the treatment or examination site 101, 201, 301 with an exemplary anatomy 102, 202, 302 illustrated therein and an instrument 103, 203, 303 for treating the anatomy site. It can be seen from FIGS. 3a-3c that areas with reduced useful information content, i.e., with no or little information content about the anatomy 102, 202, 302 being treated, are particularly suitable as target areas for embedding the further data in the video images, depending on the medical intervention performed.

For example, FIG. 3a shows an individual image 101 which may originate from an endoscopic video camera and may be confined to a circular area of the display. In this case, the entire peripheral region 104 surrounding the individual image 101 is suitable for embedding the further data and video image data.

FIG. 3b shows an individual generated video image 201, which essentially occupies the entire display area of a display device. Reserved areas 204 are shown, which are located near the corners of the video image 200 and are intended to display labels, patient information, the time of day, or other pertinent information regarding the medical intervention. One or more such reserved areas 204 may be selected for embedding the further data.

FIG. 3c shows an example in which the image edge 304 around the individual image 301 can be used as a target area for embedding the further data.

In either case, the further data may be stored in the form of a single contiguous strip, rectangle, or the like at a single location or at multiple locations distributed throughout the areas 104, 204, 304.

Returning to FIG. 2, it is illustrated that the method 31 further comprises the step S4 of modifying the video image data by embedding the further data into the video image data synchronously in time. In particular, the video image data in the identified target areas of a frame is replaced by the further data associated with the respective frame. I.e., the modified video image data in the respective target areas now contains the further data instead of the originally acquired video image data. The further data can be embedded in the video image as raw data or, alternatively, encrypted before embedding and/or provided with a digital certificate to ensure authenticity.

An essential aspect of the invention is the time-synchronous embedding of highly dynamic measurement data, e.g., the measured electrical RF readings, which are acquired by sensors or determined during a medical interventional procedure. As mentioned, these can be, for example, RF voltage, RF current, power, power factors, spark intensities and other measured values that are measured or determined at an update rate that is several times, at least twice or even plural times, greater than the frame rate of the video image data. For example, the update rate can be at least 150 Hz and preferably 250 Hz or more. In any case, this highly dynamic measurement data is acquired faster than the frame rate. For example, with an update rate of 250 Hz and a frame rate of 50 Hz, five measurement values can be stored per frame. Thus, a time course with 5 or more values of the dynamic measurement data can be recorded for each individual image and stored time-synchronously in the video image data, assigned to each associated frame.

FIG. 4 illustrates the embedding of dynamic data in the individual video frames in a simplified schematic diagram. The upper graphic in FIG. 4 shows a sequence of video frames 400 resulting from the video image data recorded by a video camera during a medical interventional procedure in a time sequence.

The second from the top graph of FIG. 4 shows an example of an electrical RF quantity 401, such as the RF current or the RF voltage, which is applied to the medical device. As can be seen, the electrical quantity 401 changes so rapidly that the amplitude fluctuates at high frequencies during a frame period.

As can be seen from the third from the top graph of FIG. 4, the electrical quantity 401 is measured or determined at an update rate that is higher than the frame rate. As a result, several, e.g., five or more, measured values 402, which are associated with the respective video frame 400, are obtained per frame period.

The bottom graphic in FIG. 4 illustrates how the acquired dynamic measurement data 403 is integrated into each video frame 400 in order to obtain a time-synchronous embedding of the dynamic measurement values 402 in the associated video frames 400.

It should be noted that in FIG. 1, the embedding of the dynamic measurement values 402 in a contiguous region in a top edge area of the video frame images is only exemplary. Various areas of different shapes can be used to store the dynamic measurement values, as explained above. Furthermore, in addition to the dynamic measurement values, other data, such as device data, are also integrated into the individual frames 400.

Returning to FIG. 2, the method further comprises the step S7 of storing the modified video image data with the further data embedded in the video image frames in a storage device, e.g., the external storage device 21 or the HIS or PACS 23 in the system according to FIG. 1 for short- to long-term archiving of the data to make it available for later analysis and evaluation. The method 31 according to the invention can then end after step S7.

In general, the video image data is stored in a compressed form in step S7. In order to obtain the smallest possible amount of data, lossy compression algorithms, such as common methods according to H.265, AV1 or VP9, or other known compression methods are usually used. In order that the further data can be extracted from the stored modified video image data and reconstructed without errors, it may be useful to subject the modified video image data obtained in optional step S5 to a compression invariance enhancement procedure which increases the reliability of a reconstruction of the further data after decompression. For this purpose, the further data can be combined, for example, into several contiguous pixels, in particular pixel blocks with, for example, 8×8, 16×16 or even up to 64×64 pixels. It is known that such coarser structures in an image suffer less compression artifacts during compression. A block formation can thus increase robustness against compression artifacts and coding errors.

As a further procedure, an error correction procedure can additionally or alternatively be used. Such error correction procedures, which are generally used to identify errors in the storage and transmission of data and to correct them as far as possible, are generally known. They may be used here to encode the further data for embedding in the video image data, in order to detect and, if necessary, correct an encoding error or a compression artifact present after the decompression.

Subsequently, the further data processed by the procedure according to step S5 may be embedded in the video image data and the resulting modified video image data may be subjected to compression according to one of the above-mentioned common techniques in step S6 in order to obtain compressed modified video image data, which is then stored in step S7. It is obvious that the processing for compression invariance enhancement according to step S5 may be performed before or after step S4 or at the same time.

It is also advantageous if steps S4, S5 and S6 are all performed in a common signal processing device, such as the signal processing device 11 of the system 1 according to FIG. 1. This device can then perform the compression invariance enhancement procedure according to step S5 as a function of the selected compression method and, in particular, already check prior to storing the resulting data whether the data can be stored correctly or whether the structure size of the data should be dynamically adapted to the compression to enable correct reconstruction of the further data.

The invention enables a subsequent analysis of the recorded video image and further data, including highly dynamic measurement data, and evaluation thereof for different goals. In particular, the medical intervention performed can be tracked and evaluated by the attending physician, the hospital or the patient, as required, in order to verify the success or failure of the intervention. The analysis can also be used to assess the performance of the medical device. For example, the input variables set for the medical intervention, the selected application modes and the effectiveness of the desired treatment effects can be assessed in all phases of the medical intervention by comparing the video image data and the associated further data. For example, spark formation and intensity can be detected visually and compared with associated electrical measured data. In this way, time-critical processes, such as the ignition behavior or a spark breakaway, can be accurately evaluated based on the embedded electrical RF measurement data. Since these processes occur much faster than a refresh rate or frame rate, acquiring the dynamic electrical measurement values over time with multiple readings per frame is extremely useful for evaluating such time-critical processes. Based on the evaluation results, optimization options for the set variables, application modes and the medical device or devices can be sought. Inadequacies, such as the limits of a setting for the respective application, for example, an insufficient speed of the treatment effects, e.g., during cutting, coagulation, thermofusion, and the like, can also be identified and compared with the associated settings in order to find more appropriate settings. The occurrence of lateral damage, such as carbonization, onset of bleeding, can be detected and the cause of such lateral damage or other complications can be determined based on the acquired measurement data.

Many further evaluations can thus be performed using the further data embedded in the video image data, in particular by comparing the electrical measurement data with the elements or events that can be visually detected and recognized in the frames, such as spark formation, instruments or electrodes, tissue and/or transitions between different tissue types, bleeding or flushing with liquids, smoke generation or vapor formation and heat generation. The evaluation of the above-mentioned relationships can be done manually or automatically using a suitable algorithm.

Further modifications are possible within the scope of the invention. For example, the further data, including the dynamic measurement data, can be incorporated into individual bits of a pixel color of a plurality of pixels of the video image data instead of into individual pixels or pixel areas. For example, if the pixel color of each pixel is encoded with 24 bits of useful information (8 bits per color channel (R, G, B)), the color depth can be reduced to 7 bits per color channel, for example, and the remaining 3 bits per pixel (1 bit per color channel) can be used to store or encode the further data. Thus, no part of the image is completely blended, but is only slightly adjusted in color. Due to the required quality of intra-operative videos, an application in the less relevant areas, e.g., the peripheral areas or the areas of the video images reserved for labeling, patient data, time and the like, is to be preferred here as well.

A further modification of the method 31 according to the invention is shown in FIG. 5. Steps S1-S7 correspond to the steps in the embodiment of FIG. 2, so that reference is made to the explanations given there to avoid repetition.

As can be seen from FIG. 5, in the embodiment according to FIG. 5, following step S7 in which the modified video image data is stored, the method 31 comprises step S8 in which the modified video image data is retrieved from the respective memory, e.g., the storage device 21 or the HIS or PACS 23 in the system 1 according to FIG. 1.

Subsequently, in step S9, decompression of the data is performed in order to obtain decompressed data.

In step S10, the further data originally embedded into the video image data, i.e., the stored examination or treatment data, device data, diagnostic data, and measurement data, especially dynamic measurement data, is then extracted from the decompressed data.

In step S11, the extracted further data and the video image data are subjected to an analysis with one or more of the goals mentioned above, e.g., for quality investigation and assurance, for assessing the settings, application modes and treatment effects, for evaluating lateral damage and other complications and finding causes thereof, for optimization of the settings or application modes or for further development of the devices. The analysis can be manual or automated.

1. A method for supporting evaluation of a video-assisted medical interventional procedure, the method comprising:
receiving medical video image data representing video images of an examined or treated anatomy recorded by a video camera at a specific frame rate during a medical intervention;
receiving further data comprising at least one of treatment or examination, device, diagnostic and measurement data associated with the medical intervention, the further data including measured dynamic data that varies during a frame period;
identifying target areas in the video images for embedding the further data; and
modifying the video image data by embedding the further data into the video image data synchronously in time by replacing video image data in the identified target area or areas of a frame with the further data associated with the respective frame.

2. The method of claim 1, further comprising at least one of the following steps:
storing the modified video image data in a non-volatile storage device for further use, in particular evaluation, in the vicinity of the place of the medical interventional procedure; and
transmitting the modified video image data to a PACS picture archiving and communication system, a CIS/HIS hospital information system or an external server for archiving image data.

3. The method according to claim 1, wherein the frame rate is between 24 and 60 frames per second.

4. The method according to claim 1, wherein the treatment or examination data include place and time of the intervention, institution involved, persons involved, name of the patient, name of the attending physician, type of medical intervention or other data relating to the medical intervention;
wherein the device data includes a type designation of the medical device or devices used, device serial number, device parameters, default settings, selected application mode, preset limit values for variables, error and status messages from the devices or other data relating to the medical devices used;
wherein the diagnostic data includes determined spectra or classification results from an optical emission spectrometry, spectra, measured values and/or classification results from an impedance spectroscopy or from other diagnostic methods;
wherein the measurement data includes measured or determined electrical RF variables, such as voltage, current, power, power factor and/or spark intensity, parameters or variables of a neutral electrode, such as transition impedance, current symmetry and/or current density, or other measurement variables measured or determined during the medical intervention.

5. The method according to claim 1, wherein the measured dynamic data includes measurement variables that are acquired or determined at an update rate that is several times greater than the frame rate of the video image data, the update rate being at least 150 Hz.

6. The method according to claim 1, wherein measured dynamic data which characterizes a time course of two or more measured values of a measured variable, in particular a measured electrical variable, is stored in individual frames.

7. The method according to claim 1, wherein identifying target areas in the video images for embedding the further data is based on areas in the video images that are defined in advance or are specified or can be specified by a user.

8. The method according to claim 1, wherein the further data is stored at defined positions distributed over the image.

9. The method according to claim 1, wherein the further data is embedded in areas of the video images for labeling, displayed patient data, time and/or at an image edge.

10. The method according to claim 1, wherein the further data is incorporated into individual bits of a pixel color of a plurality of pixels of the image data.

11. The method according to claim 10, wherein the further data is incorporated in image areas with a lower useful information content.

12. The method according to claim 1, wherein the modified video image data is lossy compressed, wherein the modified video image data is processed by a compression invariance enhancement procedure before a compression method is performed, in order to increase reliability of a reconstruction of the further data.

13. The method according to claim 1, wherein the modification of the video image data and the compression of the modified video image data are performed in a common signal processing device, which performs the compression invariance enhancement procedure depending on the selected compression method.

14. The method according to claim 13, wherein the common signal processing device is able to dynamically adapt the structure size and/or encoding of the data to the compression.

15. The method of claim 1, further comprising the steps of:
retrieving the stored modified video image data;
extracting the further data from the modified video image data; and
analyzing the extracted static and measured dynamic data in conjunction with the video image data configured to evaluate one or more of the following assessment of the input variables set for the medical intervention, the selected application modes, the effectiveness of the desired treatment effects, finding optimization possibilities for the set variables, application modes or the medical device or devices, or detection of inadequacies.

16. The method of claim 15, wherein the analyzing the extracted static and measured dynamic data includes detection of one or more of the following inadequacies: electrical limitations for the application, insufficient speed of the treatment effects, occurrence of lateral damage, including carbonization or onset of bleeding, and, if occurrence of lateral damage is detected, further includes determination of causes of the lateral damage or other complications.

17. A system for supporting evaluation of a video-assisted medical interventional procedure, the system comprising:
at least one medical device configured to perform a medical intervention;
a video camera configured to record video image data representing video images of treated or examined anatomy during a medical intervention;
a signal processing device configured to process the video image data recorded by the video camera;
a data connection between the at least one medical device and the signal processing device configured to transmit further data, including dynamic data, from the at least one medical device to the signal processing device.

18. The system according to claim 17, wherein the signal processing device comprises a data interface in communication with a PACS picture archiving and communication system or a CIS/HIS hospital information system and/or an external server for archiving image data.
